(19) United States
(12) Patent Application Publication
(10) Pub. No.: US 2015/0253312 A1
(43) Pub. Date: Sep. 10, 2015

(54) SOLUTION CONTAINERS HAVING CONTAMINATION DETECTION AND INDICATION CAPABILITY

(71) Applicant: CIRLE, Inc., Miami, FL (US)

(72) Inventor: Richard Awdeh, Miami, FL (US)

(21) Appl. No.: 14/432,337

(22) PCT Filed: Sep. 27, 2013
(86) PCT No.: PCT/US2013/062286

§ 371 Date: Mar. 30, 2015

Related U.S. Application Data
(60) Provisional application No. 61/707,211, filed on Sep. 28, 2012.
(60) Provisional application No. 61/827,302, filed on May 24, 2013.

(51) Int. Cl. G01N 33/52; B65D 47/06; B65D 25/04; B65D 81/32; B65D 1/02; B65D 25/42

(52) U.S. Cl. CPC G01N 33/528; B65D 1/0246; B65D 25/42; B65D 25/04; B65D 81/32; B65D 47/06

(57) ABSTRACT

In one embodiment, a solution container includes a body adapted to store a solution, a nozzle through which the solution can exit the body, and a biosensor material integrated with the container, wherein the biosensor material has a first color when it has not been exposed to a contaminant but turns a second color when it comes into contact with a contaminant.

### CROSS-REFERENCE TO RELATED APPLICATION(S)

This application claims priority to co-pending U.S. Provisional Application Ser. No. 61/707,211, filed Sep. 28, 2012, and U.S. Provisional Application Ser. No. 61/827,302, filed May 24, 2013, which are both hereby incorporated by reference herein in their entireties.

### BACKGROUND

Various solutions are intended for use with the body. For example, ophthalmic solutions, such as eye drops, are intended for use on the eyes. It is possible for such solutions to become contaminated, in which case they should not be used. Unfortunately, it is typically impossible for the user of the solution to know whether or not the solution is contaminated because the contamination normally cannot be seen.

In view of the above facts, it can be appreciated that it would be desirable to have means for detecting contamination of a solution and communicating the presence of the contaminant to a user.

### BRIEF DESCRIPTION OF THE DRAWINGS

The present disclosure may be better understood with reference to the following figures. Matching reference numerals designate corresponding parts throughout the figures, which are not necessarily drawn to scale.

FIGS. 1A and 1B illustrate solution containers having a biosensor material provided on the inside surfaces of the bodies of the containers.

FIGS. 2A-2C illustrate solution containers having a biosensor material provided within the bottoms of the bodies of the containers.

FIGS. 3A-3C illustrate solution containers that contain independent biosensor material elements.

FIGS. 4A and 4B illustrate solution containers having nozzles that incorporate a biosensor material.

FIGS. 5A and 5B illustrate solution containers having nozzles that extend into the bodies of the containers and that incorporate a biosensor material.

FIGS. 6A and 6B illustrate an inverted solution container including a nozzle that incorporates a biosensor material.

FIG. 7 illustrates a solution container including a neck that incorporates a biosensor material.

FIG. 8 illustrates a solution container including a cap that incorporates a biosensor material.

FIGS. 9A and 9B illustrate an inverted solution container including a cap that incorporates a biosensor material.

FIGS. 10A and 10B illustrate a solution container having a cap that includes an external test well that contains a biosensor material.

FIGS. 11A and 11B illustrate caps for a solution container that include a multiple external test wells that contain a biosensor material.

FIGS. 12A and 12B illustrate a solution container having a base member that includes multiple test wells that contain a biosensor material.

FIG. 13 illustrates a solution container having a test chamber that contains a biosensor material.

FIGS. 14A-14C illustrate solution containers that include test strips that incorporate a biosensor material.

FIG. 15 illustrates a packaging system that includes a solution container and an insert that incorporates a biosensor material.

FIGS. 16A-16C illustrate a solution container and various safety collars that can be provided on the container, the safety collars incorporating a biosensor material.

FIG. 17 illustrates a solution container having a biosensor material provided on the exterior of a body of the container.

### DETAILED DESCRIPTION

As described above, it would be desirable to have means for detecting contamination of a solution and communicating the presence of the contaminant to a user. Disclosed herein examples of such means. In some embodiments, the means are provided on or in relation to a solution container and can both detect and provide a visual indication of the contamination so that the user will know not to use the solution. In some embodiments, the solution comprises an ophthalmic solution intended for use on the eyes. More generally, however, the solution can comprise any liquid that could become contaminated.

In the following disclosure, various specific embodiments are described. It is to be understood that those embodiments are example implementations of the disclosed inventions and that alternative embodiments are possible. All such embodiments are intended to fall within the scope of this disclosure.

Described in the following disclosure are solution containers or elements associated with solution containers that comprise a biosensor material that provides an indication of the presence of a contaminant. As used herein, the term “contaminant” is an inclusive term that refers to any substance that is not intended to be present and, such as pathogens, microbial contaminants, bacterial contaminants, viral contaminants, amoebal contaminants, organic material, analytes, or combinations thereof. In some embodiments, the biosensor material changes color when it comes into contact with the contaminant. For example, the biosensor material can have an initial color (e.g., blue) and can change to a warning color (e.g., red) if and when it comes into contact with a contaminant. In such a case, the presence of the contaminant can be clearly communicated to the user as a warning.

The biosensor material that is used can depend upon the particular application. In some embodiments, however, the biosensor material comprises a polydiacetylene (PDA) polymer, which is formed by the 1,4 addition of diacetylenic monomers. When PDA polymer is exposed to ultraviolet irradiation it adopts a deep blue color. When the polymer is exposed to a contaminant, it turns red. As used herein, the term “biosensor material” includes the material that changes color in the presence of a contaminant and any other material that are mixed with the color-change material in order to enable its use on or in association with a solution container.

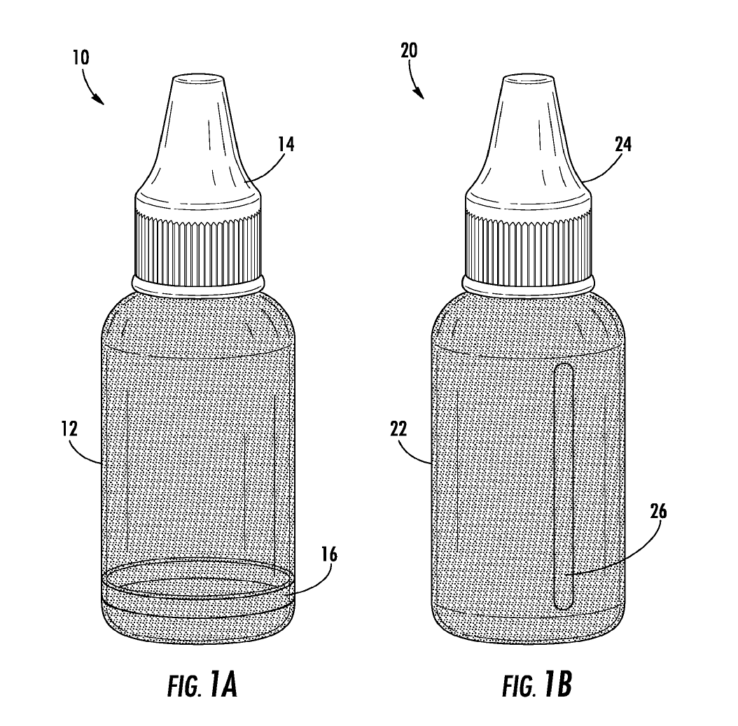

In some embodiments, the biosensor material can be incorporated into the solution container. FIGS. 1A and 1B illustrate two examples of such incorporation. Beginning with FIG. 1A, a solution container 10 takes the form of a generally cylindrical bottle having a body 12 and a cap 14, both of which can be made of a polymeric material. The body 12 is adapted to contain the solution, which can be dispensed from the body through a nozzle (not visible) that is covered by the cap 14. Provided on the inside surface (e.g., inner wall) of the body 12 near its base is a circular ring 16 of biosensor material that is in continuous contact with the solution contained by the body.

The biosensor material can have an initial color that matches the color of the remainder of the body 12. For example, if the biosensor material has a blue initial color, the body 12 can likewise be colored a similar shade of blue. Regardless, the biosensor material can have a warning color that contrasts the color of the body 12. In such a case, it will be easy for the solution user to determine when the solution has become contaminated.

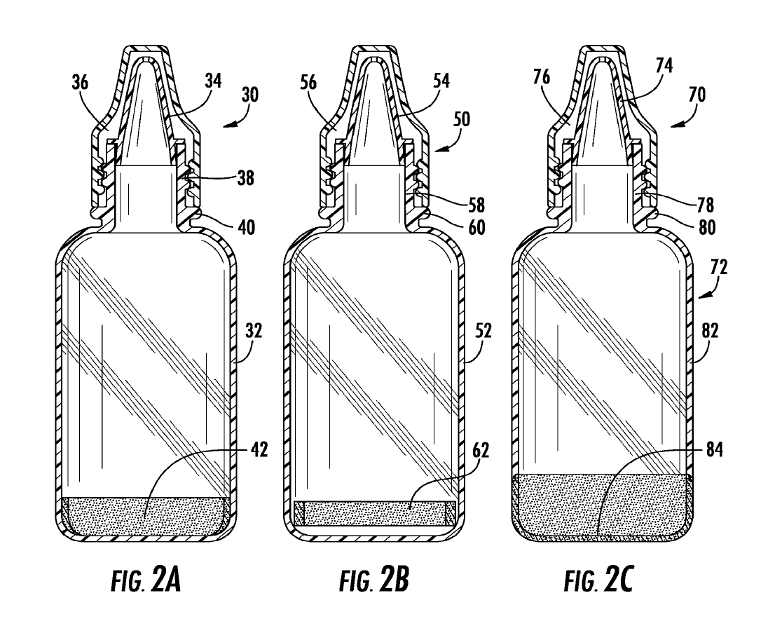

In some embodiments, the ring 16 of biosensor material can be formed by embedding the material into the polymeric material used to form the body 12 during its fabrication. In other embodiments, the biosensor material can be applied to a substrate that is adhered to the inside surface of the body 12 or can be sprayed onto the inside surface of the body (see FIG. 2).

FIG. 1B illustrates a variation on the embodiment of FIG. 1A. In particular, FIG. 1B illustrates a solution container 20 in the form of a bottle having a body 22 and a cap 24 in similar manner to the container 10 of FIG. 1A. However, instead of having a ring of biosensor material provided near the base of the body 22, the container 20 has a vertically aligned linear strip 26 of biosensor material that extends along the inside of the body from a position near its base to a position near its neck (i.e., along a length direction of the body). As with the embodiment of FIG. 1A, the biosensor material can have an initial color that matches the color of the remainder of the body 22 but can change to a contrasting color when it comes into contact with a contaminant.

FIGS. 2A-2C illustrate further examples of a biosensor material incorporated into a solution container. Beginning with FIG. 2A, shown is a solution container 30 that takes the form of a generally cylindrical bottle having a body 32, a nozzle 34, and a cap 36, each of which can be made of a polymeric material. As shown in the figure, the body 32 includes a threaded neck 38 to which the nozzle 34 is mounted (e.g., using a press fit) and onto which the cap 36 threads. Positioned below the threads of the neck 38 is a collar 40 that the cap 36 can abut when fully threaded onto the neck.

The body 32 is adapted to contain a solution, which can be dispensed from the body through the nozzle 34, for instance, when the body is squeezed. In some embodiments, body 32 is made of a clear polymeric material. Provided on the inside surface (e.g., inner wall) of the body 12 near its base is a coating 42 of biosensor material that is in continuous contact with the solution contained by the body. In some embodiments, the coating 42 can be applied by spraying the biosensor material onto the inner surface of the body 12 with a micropipette equipped with a micronozzle (not shown).

FIG. 2B illustrates a similar solution container 50. Like the container 30, the container 50 takes the form of a generally cylindrical bottle having a body 52 that comprises a threaded neck 58 and a collar 60, a nozzle 54, and a cap 56. The body 52 is adapted to contain a solution that can be dispensed from the body through the nozzle 54 when the body is squeezed. Unlike the container 30, the body 52 of the container 50 contains an independent circular or cylindrical ring 62 of biosensor material. In some embodiments, the ring 62 comprises a substrate made of glass or a thermoplastic material, such as poly(methyl methacrylate) (PMMA), to which the biosensor material is applied or in which the biosensor material is embedded. Alternatively, the ring 62 can be solely composed of the biosensor material. Regardless, the ring 62 is shaped and sized to seat within the bottom of the body 52 with a friction fit so that it will not move from the base of the body when the body is inverted.

FIG. 2C illustrates another similar solution container 70. Like the containers 30 and 50, the container 70 has the form of a generally cylindrical bottle comprising a body 72 that includes a threaded neck 78 and a collar 80, a nozzle 74, and a cap 76. Unlike the other containers, however, the body 72 comprises two independent parts, namely, an upper portion 82 and a lower portion 84. In the illustrated example, the upper portion 82 forms the majority of the body 72 while the lower portion 84 generally forms the base of the body. Regardless, the two portions 82, 84 can be connected together in a manner in which no fluid can pass into or out of the container 70 at the joint between the two portions. In some embodiments, the lower portion 84 can be snap-fit onto the upper portion 82. In other embodiments, the two portions 82, 84 can be welded or adhered together.

Irrespective of the nature of the connection between the upper and lower portions 82, 84 of the body 72, the lower portion comprises biosensor material that is in continuous contact with the solution contained in the body 72. In some embodiments, the biosensor material is embedded in the material used to form the lower portion 84. In other embodiments, the biosensor material can be applied to the inner surface of the lower portion 84 as a coating.

In each of the embodiments of FIG. 2, the biosensor material can have an initial color when no contaminants are present and can change to a warning color when the biosensor material comes into contact with a contaminant. This color change can be easily seen because the bodies of the containers are clear. Therefore, the user can be alerted when the solution has been contaminated.

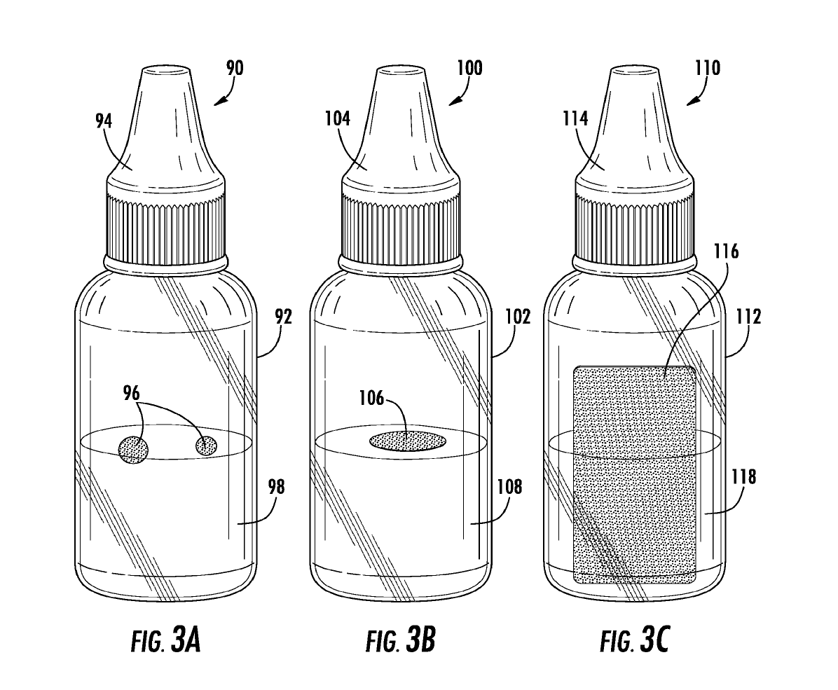

FIGS. 3A-3C illustrate embodiments of solution containers that incorporate independent biosensor material elements that can float or sit within the solution. Beginning with FIG. 3A, a solution container 90 takes the form of a generally cylindrical bottle having a body 92 and a cap 94. As before, the body 92 can be made of a clear polymeric material that enables a user to see within the body. Provided within the body 92 are one or more balls 96 of biosensor material that can float on the surface of the solution 98. In some embodiments, the balls 96 are composed solely of the biosensor material and no substrate is needed. In other embodiments, the balls 96 comprise a substrate to which the biosensor is applied or in which the biosensor is embedded.

FIG. 3B shows a further solution container 100 that takes the form of a generally cylindrical bottle having a body 102 and a cap 104. Instead of balls of biosensor material, provided within the body 102 are one or more thin films 106 of biosensor material that can float on the surface of the solution 108. In some embodiments, the films 106 are composed solely of the biosensor material and no substrate is needed. In other embodiments, a substrate is used.

FIG. 3C illustrates a variation on the embodiment shown in FIG. 3B. In FIG. 3C, a solution container 110 takes the form of a generally cylindrical bottle having a body 112 and a cap 114, and a thin film 116 of biosensor material is contained within the body. In this embodiment, however, the film 116 sits upright within the body 112 so as not to float on the surface of the solution 118. As before, the film 116 can be composed solely of the biosensor material or can include a substrate.

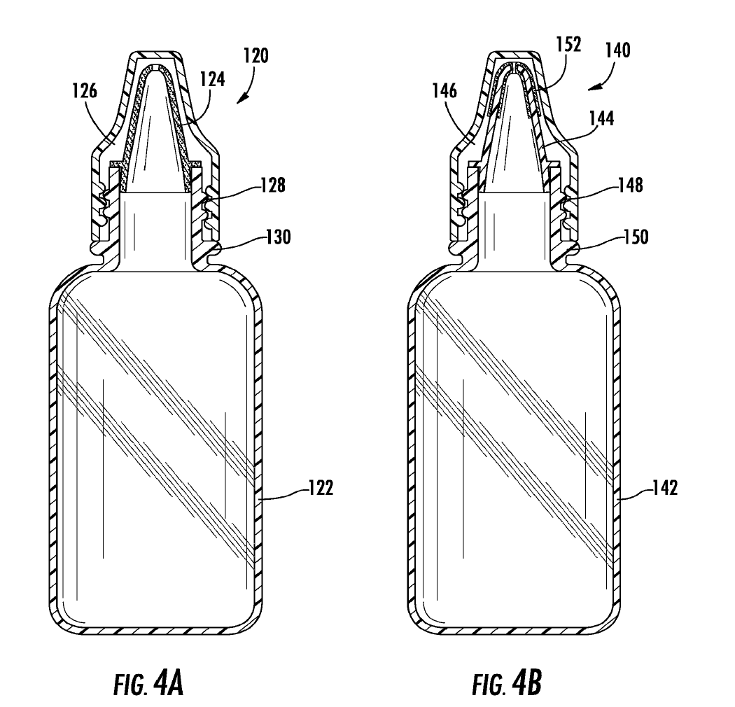

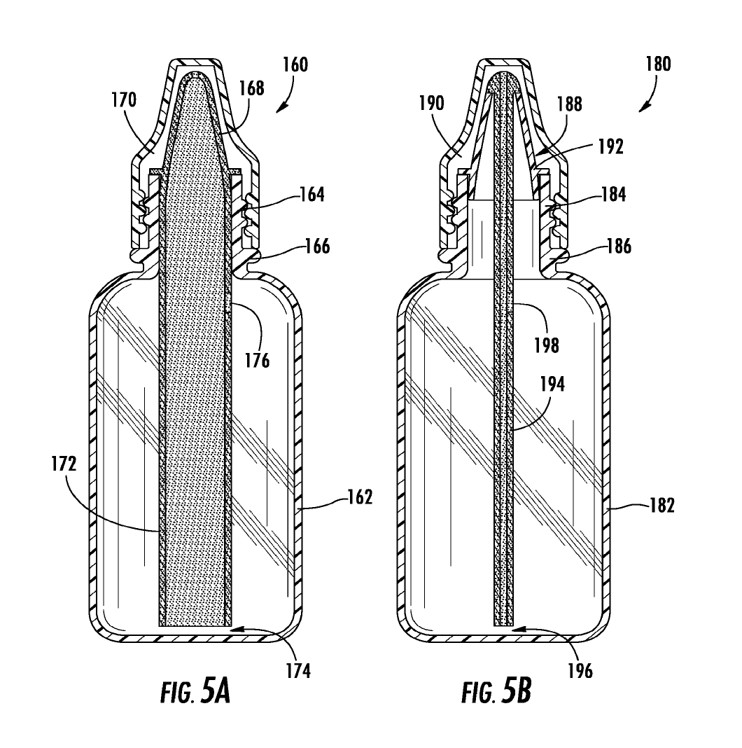

The biosensor material can alternatively be incorporated into the nozzle of a solution container. FIGS. 4A and 4B illustrate examples of such embodiments. Beginning with FIG. 4A, a solution container 120 is configured as a generally cylindrical bottle comprising a body 122 including a threaded neck 128 and a collar 130, a nozzle 124, and a cap 126, each of which can be made of a polymeric material.

In the embodiment of FIG. 4A, the biosensor material is embedded into the nozzle 124, which can be composed of a clear polymeric material. In such an arrangement, the biosensor material is not in continuous contact with the solution contained in the body 122 but comes into contact with the solution when the container 120 is inverted and/or when solution is dispensed from the container via the nozzle 124.

FIG. 4B illustrates a similar embodiment. In this figure, a solution container 140 comprises a body 142 including a threaded neck 148 and a collar 150, a nozzle 144, and a cap 146, each of which can be made of a polymeric material. Instead of the biosensor material being embedded into the material of the nozzle 144, however, the biosensor material is applied to the inner and/or outer surfaces of the nozzle as a coating 152. In some embodiments, the coating 152 can be formed using a dip-coating process. As with the embodiment of FIG. 4A, the biosensor material is not in continuous contact with the solution contained in the body 142 but comes into contact with the solution when the container 140 is inverted and/or when solution is dispensed from the container via the nozzle 144.

FIGS. 5A and 5B illustrate embodiments in which the nozzle incorporating a biosensor material comprises an insert that extends down into the body container. Beginning with FIG. 5A, illustrated is a solution container 160 arranged as a generally cylindrical bottle comprising a body 162 that includes a threaded neck 164 and a collar 166, a nozzle 168, and a cap 170. The body 162 can be clear so as to enable a user to see into the body. As shown in the figure, the nozzle 168 is inserted into the neck 164 of the body 162 but comprises an elongated member 172, such as a cylindrical tube, that extends deep into the body. As shown in FIG. 5A, the member 172 can extend down to the base of the body 162 so that it nearly contacts the bottom inner surface of the body. Because there is a gap 174 between the bottom end of the member 172 and the base of the body 162, solution can enter the interior of the member and flow out through the tip of the nozzle 168. In addition, one or more openings 176 can be formed in the member 172 near the neck 164 of the body 162 to enable the solution to exit the container 160.

As is depicted in FIG. 5A, the entire nozzle 168, including the elongated member 172, can comprise a biosensor material. In some embodiments, the biosensor material is embedded within the polymeric material used to form the nozzle 168. In other embodiments, the biosensor material can be coated on the nozzle 168.

FIG. 5B illustrates a solution container 180 that also comprises a body 182 including a threaded neck 184 and a collar 186, a nozzle 188, and a cap 190. In this embodiment, however, the nozzle 188 comprises a first portion 192 that directly connects to the neck 184 of the body 182 and a second portion 194 that extends down from the first portion deep into the body. The second portion 194 can also be configured as an elongated member, such as a cylindrical tube, and can form a gap 196 with the bottom inner surface of the body 182. The second portion 194 can also include or more openings 198 can be formed near the neck 184 of the body 182 to enable the solution to exit the container 180.

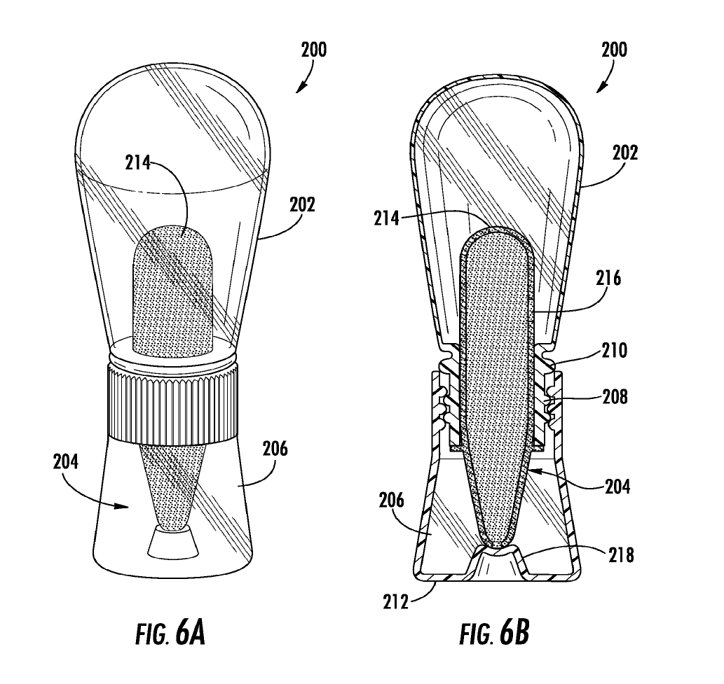

FIGS. 6A and 6B illustrate another solution container 200 including a nozzle that incorporates a biosensor material. As shown in these figures, the container 200 comprises a bulbous body 202, a nozzle 204, and a cap 206, each of which may be made of a polymeric material. In some embodiments, at least the body 202 and the cap 206 are made of a clear polymeric material so that the user can see through them. The body 202 comprises a threaded neck 208 and a collar 210 and, as with other embodiments described above, the cap 206 threads onto the neck. Unlike the other embodiments, the container 200 has an inverted configuration in which the nozzle 204 faces downward and the cap 206 serves as a support or base for the body 202. As shown in FIG. 6B, the cap 206 comprises a planar bottom surface 212 to enable this functionality. As is further shown in the figure, the cap 206 can have a generally frustoconical shape.

As shown most clearly in FIG. 6B, the nozzle 204 can, like the embodiments of FIGS. 5A and 5B, include an elongated member 214 that extends into the body 202 so that it is visible through the walls of the body. The member 214 can also include openings 216 that enable the solution contained in the body 202 to exit through the tip of the nozzle 204. In some embodiments, the entire nozzle 204, including the member 214 comprises biosensor material. For example, the biosensor material can be embedded within the polymeric material used to form the nozzle 204 or the biosensor material can be coated on the nozzle. In either case, color change of the biosensor material can be easily seen through the clear body 202 and/or cap 206.

In some embodiments, the container 200 can further include a stopper 218 in the form of a conical element provided within the cap 206 that extends up to the tip of the nozzle 204 and prevents solution from leaking out from the body via the nozzle.

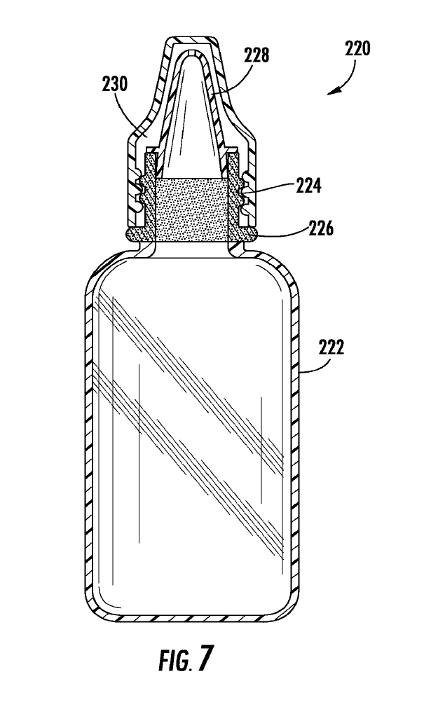

With reference next to FIG. 7, illustrated is a solution container 220 having the form of a generally cylindrical bottle comprising a body 222 including a threaded neck 224 and a collar 226, a nozzle 228, and a cap 230, each of which can be made of a polymeric material. In this embodiment, it is the neck 224 and collar 226 of the body 222 that incorporate the biosensor material. In some embodiments, the biosensor material can be embedded in the clear polymeric material used to form the neck 224 and collar 226. In other embodiments, the neck 224 and collar 226 can be coated with the biosensor material. In embodiments in which the cap 230 is clear, both the neck 224 and collar 226 are visible when the cap is screwed on. In embodiments in which the cap 230 is opaque, at least the collar 226 is visible when the cap is screwed on.

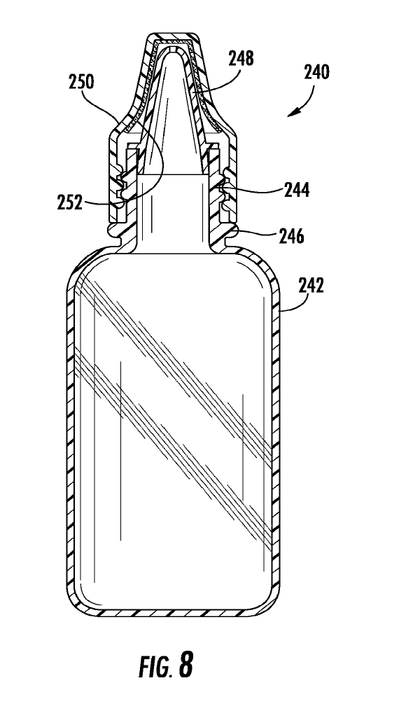

A biosensor material can also be incorporated into a cap of a solution container. FIG. 8 illustrates an example of this. In particular, FIG. 8 illustrates solution container 240 comprising a body 242 including a threaded neck 244 and a collar 246, a nozzle 248, and a cap 250, each of which can be made of a polymeric material. In some embodiments, the cap 250 can be made of a clear polymeric material.

As shown in FIG. 8, a coating 252 comprising a biosensor material overlies an inner surface of the cap 250 near the nozzle 248 (when the cap is screwed on) so as to form an inner test well. In such an embodiment, the solution within the body 242 can be tested by the user by squeezing out a drop of material onto the coating 252 provided within the cap 250. If the coating changes color, the user knows that the solution is contaminated.

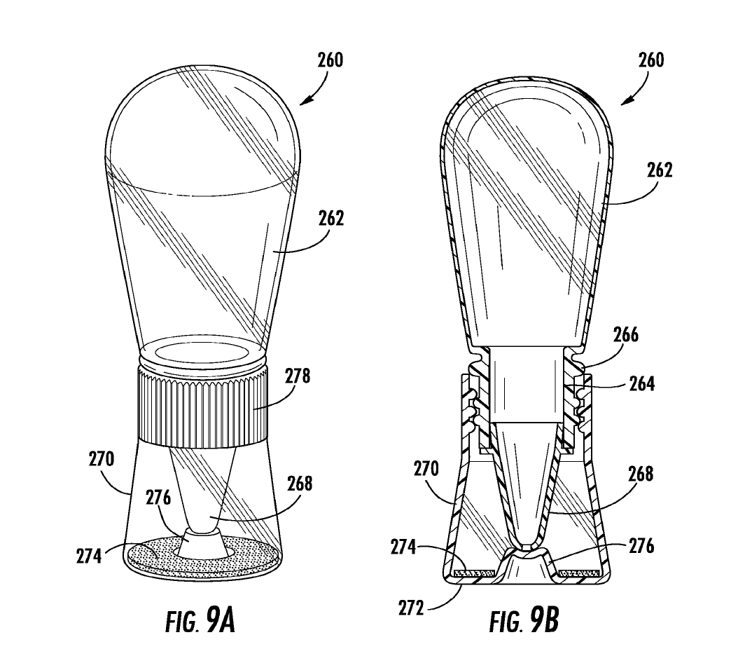

FIGS. 9A and 9B illustrate another solution container having a cap that incorporates a biosensor material. More particularly, FIGS. 9A and 9B illustrate a solution container 260 that, like the container 200 of FIGS. 6A and 6B, has an inverted configuration. The container 260 comprises a bulbous body 262 that includes a threaded neck 264 and a collar 266, a nozzle 268, and a cap 270, each of which may be made of a polymeric material. In some embodiments, at least the cap 270 is made of a clear polymeric material so that the user can see through it. In order to be able to support the body 262, the cap 270 comprises a planar bottom surface 272. As is further shown in the figures, the cap 270 can have a generally frustoconical shape.

Provided at the bottom inner surface of the cap 270 is a layer 274 of biosensor material so as to form an inner test well. In some embodiments, the layer 274 can be sprayed on the inner surface of the cap 270. In other embodiments, the layer 274 can comprise an insert that is positioned at the bottom of the cap 270. Regardless, the solution can be tested by the user by squeezing a drop of the solution onto the layer 274 to see if it will change color.

As is further shown in FIGS. 9A and 9B, the cap 270 can further comprise a stopper 276 in the form of a conical element that extends up to the tip of the nozzle 268 and prevents solution from leaking out from the body 262 via the nozzle. In such cases, the layer 274 of biosensor material is a ring of biosensor material that has a central opening and surrounds the stopper 276. With particular reference to FIG. 9A, the cap 270 can further have a knurled outer surface 278 that enables the user to better grip the cap.

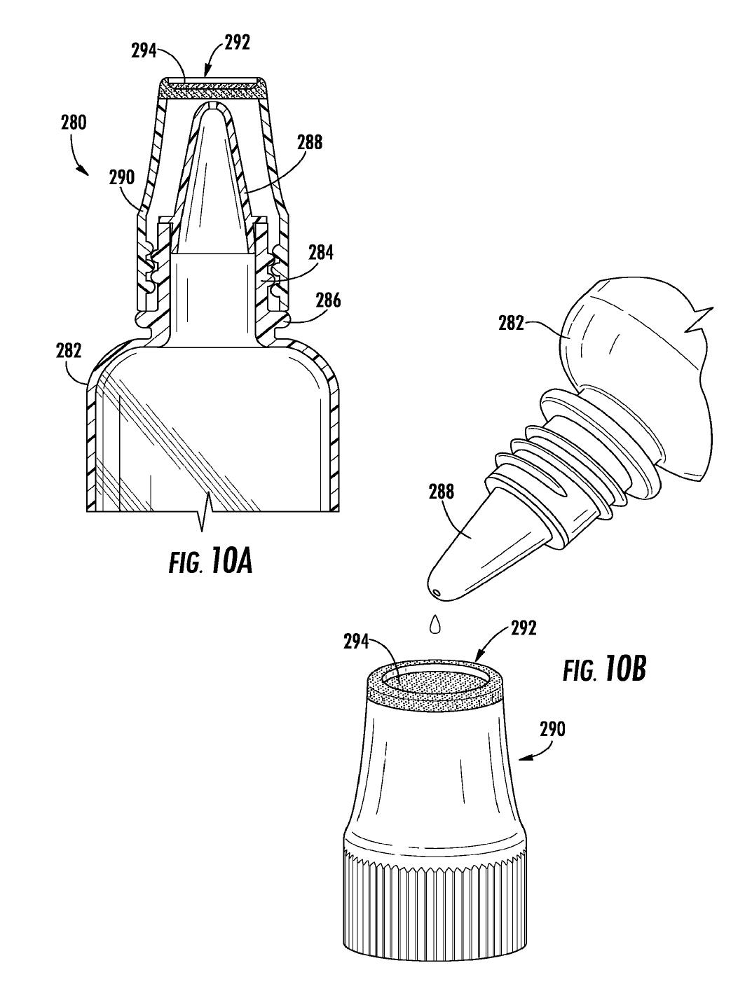

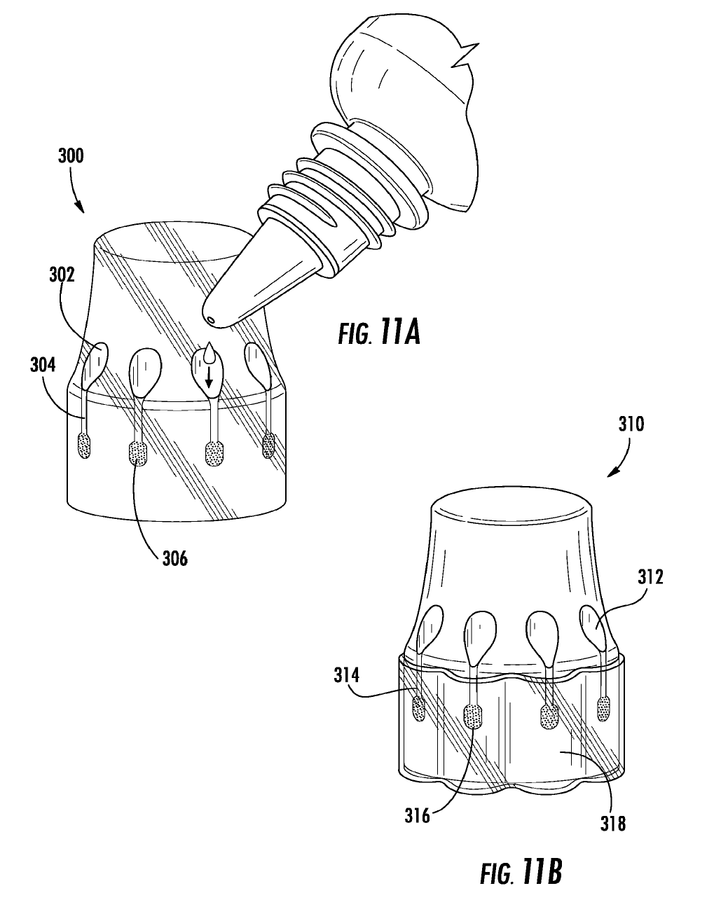

FIGS. 10 and 11 illustrate embodiments in which a cap of a solution container comprises one or more external test wells that hold a biosensor material. Beginning with FIGS. 10A and 10B, a solution container 280 comprises a body 282 including a threaded neck 284 and a collar 286, a nozzle 288, and a cap 290, each of which can be made of a polymeric material. As shown in the figures, the cap 290 includes an external well 292 provided on its top in which a layer 294 of biosensor material is provided. With such a configuration, the solution within the body 282 can be tested by the user by squeezing out a drop of material onto the layer 294 of biosensor material provided within the well 292, as depicted in FIG. 10B. If the coating changes color, the user knows that the solution is contaminated.

In some embodiments, a top portion of the cap 290 can have a color that is similar to the color of the biosensor material in its initial state. In such a case, the biosensor material's color will strongly contrast that of the top of the cap 290 if a contaminant is detected.

FIGS. 11A and 11B illustrate two alternative container caps 300 and 310, respectively, that comprise multiple external wells. Beginning with FIG. 11A, the cap 300 comprises multiple openings 302 provided around the outer periphery of the cap in which the solution can be dropped. The cap 300 can be constructed of a clear polymeric material. As shown in FIG. 11A, each opening 302 is in communication with an internal channel 304 that leads to an internal cavity 306 formed within the cap 300 in which a biosensor material is provided. In such an embodiment, a droplet of solution can be dropped into one of the openings 302, flow down to through the channel 304 associated with the selected opening, and mix with the biosensor material contained within an internal cavity 306 associated with the channel. If the solution is contaminated, the biosensor material, which is viewable through the clear cap 300, will change color. Multiple openings 302 and cavities 306 are provided for cases in which the biosensor material cannot be reused.

Referring next to FIG. 11B, the cap 310 also comprises multiple openings 312 in communication with internal channels 314 that lead to internal cavities 316 in which a biosensor material is provided. Accordingly, the cap 310 can be used to test solution in similar manner to that described above in relation to FIG. 11A. The cap 310, however, further includes magnifying lenses 318 associated with each internal cavity 316 that magnify the biosensor material so that the user can see the color change more easily. In some embodiments, the lenses 318 are part of an outer ring element that surrounds the cap 310.

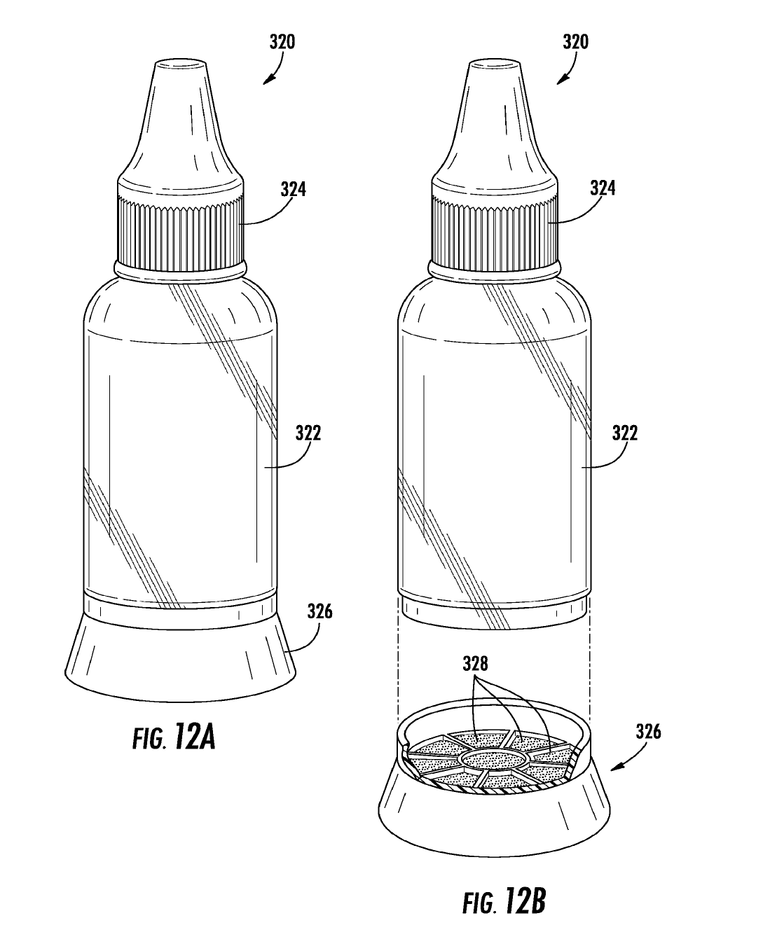

FIGS. 12A and 12B illustrate a further embodiment that incorporates test wells. As shown in these figures, a solution container 320 comprises a body 322 and a cap 324, both of which can be made of a polymeric material. The container 320 further comprises a base member 326 upon which the body 322 can rest, as indicated in FIG. 12A. When the body 322 is picked up off of the base member 326, however, multiple test wells 328 provided on the top of the base member become accessible. Each of the wells 328 can include a layer of biosensor material. In such an embodiment, a droplet of solution can be dropped into one of the wells 328 to see if it will invoke a color change in the biosensor material.

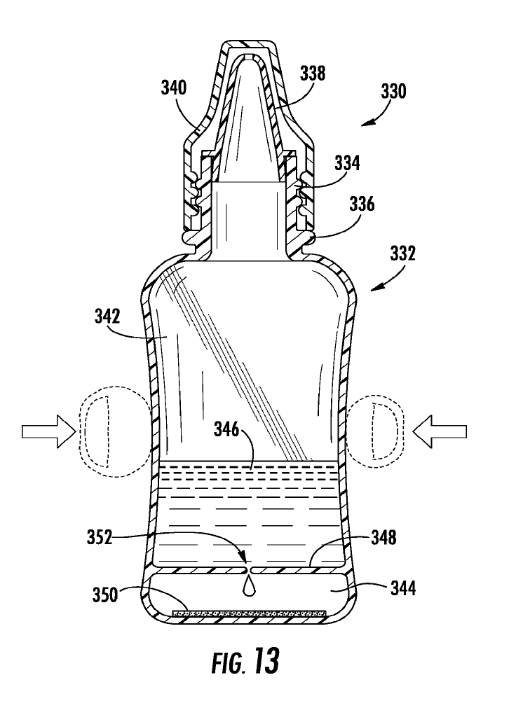

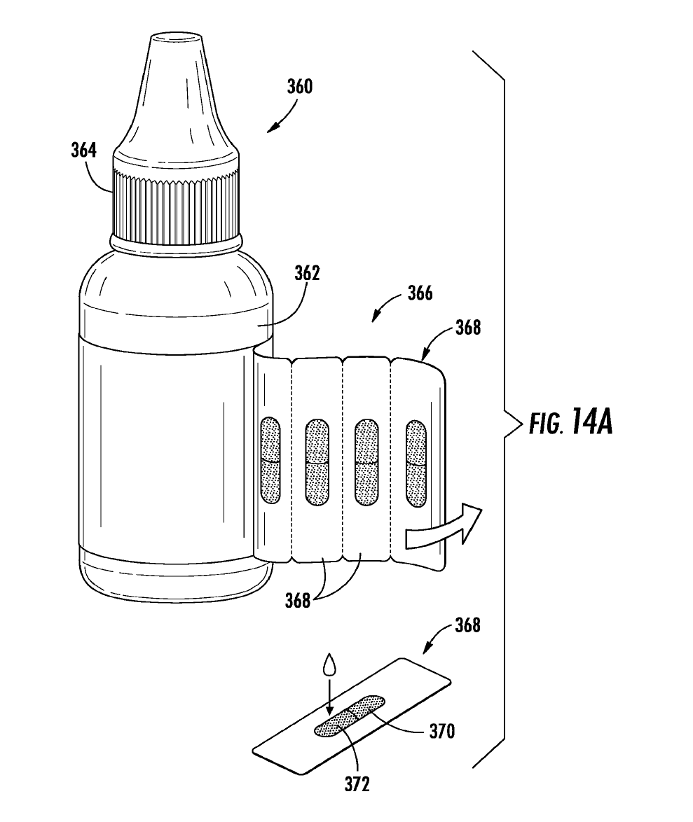

In other embodiments, a testing well can be integrated into the body of a solution container. Such an embodiment is shown in FIG. 13, which illustrates a solution container 330. Like several of the other disclosed embodiments, the container 330 comprises a body 332 including a threaded neck 334 and a collar 336, a nozzle 338, and a cap 340, each of which can be made of a polymeric material. Unlike previous embodiments, however, the body 332 comprises two independent chambers, including a first or upper chamber 342 and a second or bottom chamber 344 that serves as a test chamber. The upper chamber 342 contains the solution 346, which can exit the body 332 via the nozzle 338 as in the other embodiments. The lower chamber 344 is positioned below the upper chamber 342 and is separated therefrom by a dividing wall 348. The lower chamber 344 contains air and a layer 350 of biosensor material that is provided on the bottom inner surface of the chamber.

The solution 346 in the upper chamber 342 can be tested by transferring one or more droplets of solution from the upper chamber to the lower chamber 344 through a small one-way valve 352 provided in the dividing wall 348. To do this, the user simply squeezes the body 332 while in an upright orientation with the cap 340 affixed. This action increases the pressure within the upper chamber 342 and forces one or more droplets through the valve 352 and onto the biosensor material, which will change color if the solution is contaminated.

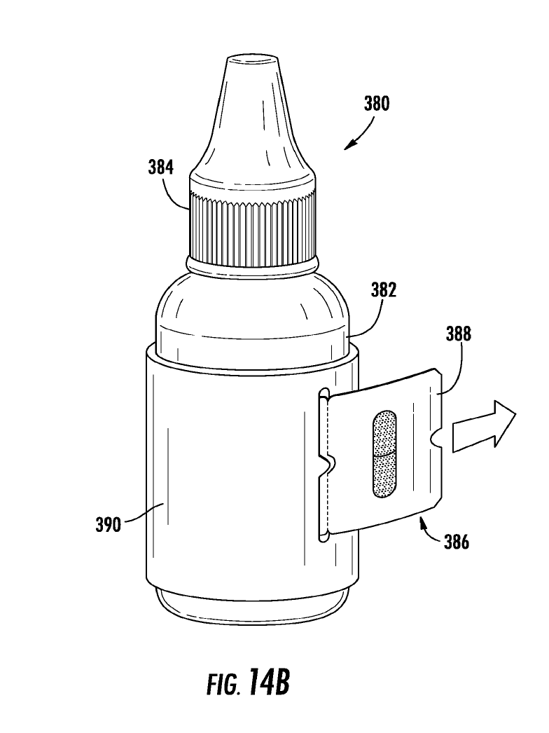

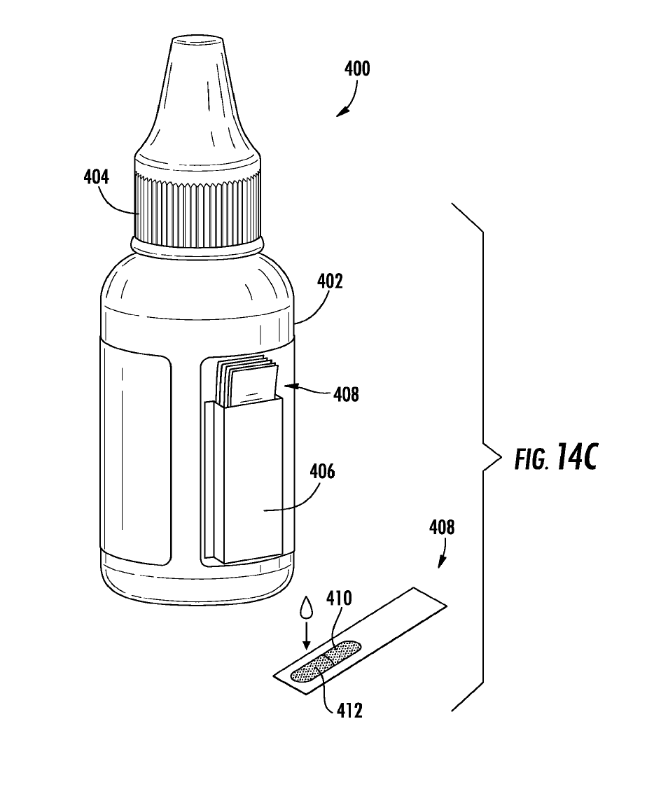

In some embodiments, the biosensor material can be provided on object that is separate from but associated with the solution container. FIGS. 14-16 illustrate examples of such embodiments. Beginning with FIG. 14A, illustrated is a solution container 360 that comprises a body 362 and a cap 364. Wrapped around the body 362 is a continuous band 366 of material, such as paper. In some embodiments, the band 366 is lightly adhered to the body 362 so that it can be pulled off from the body. As shown in FIG. 14A, the band 366 comprises multiple test strips 368 that are defined by perforated edges so that they may be individually torn from the band. Each test strip 368 comprises a biosensor material that can be used to test the solution within the body 362 of the container 360. In some embodiments, each test strip 368 comprises a control area 370 and a biosensor area 372. The control area 370 can have a color similar to the color of the biosensor area 372 when in its initial state (not exposed to contaminant). In such a case, the color of the biosensor area 372 can be compared to the color of the control area 370. If they match after the solution is applied to the biosensor area 372, the solution is not contaminated. If they do not match (e.g., significantly contrast each other) after the solution is applied to the biosensor area 372, the solution is contaminated.

FIG. 14B illustrates a solution container 380 that also comprises a body 382, a cap 384, and a continuous band 386 of material, such as paper, that comprises multiple test strips 388 that are defined by perforated edges. As above, each test strip 388 comprises a biosensor material that can be used to test the solution within the body 382 of the container 380. In some embodiments, each test strip 368 also comprises a control area 370 and a biosensor area 372. In the embodiment of FIG. 14B, however, the band 386 is contained in a sleeve 390 that is formed around the outer periphery of the body 382.

FIG. 14C shows another solution container 400 that comprises test strips. In this embodiment, the container 400 comprises a body 402 and a cap 404. Provided on the body 402 is an outer pocket 406 in which multiple test strips 408 can be stored for later individual use. Again, each test strip 408 comprises a biosensor material that can be used to test the solution within the body 402 of the container 400. In some embodiments, each test strip 408 comprises a control area 410 and a biosensor area 412 so that the solution can be evaluated by comparing the colors of the two areas after a droplet of the solution has been applied to the biosensor area.

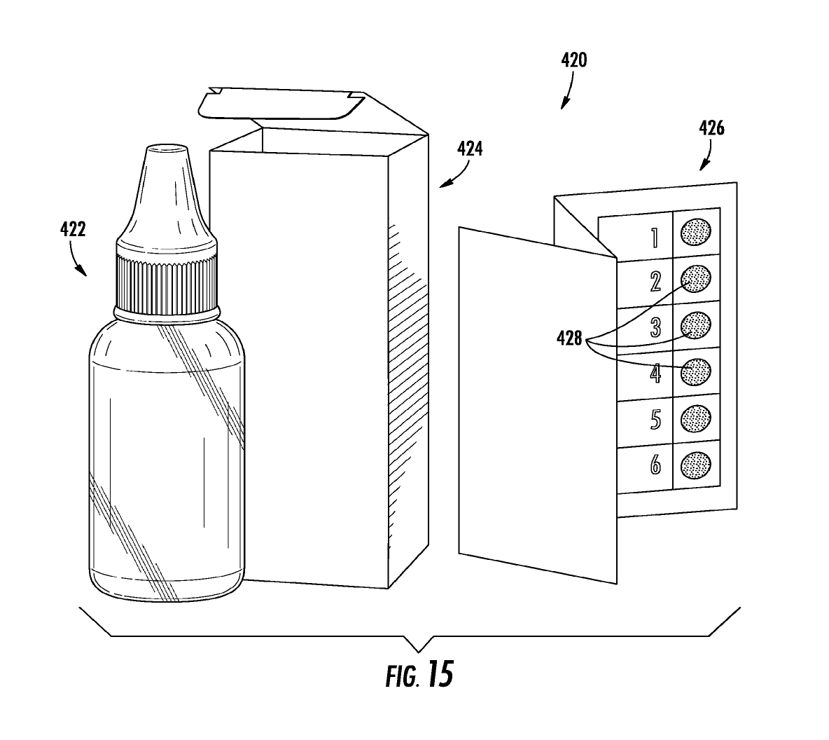

In some cases, test strips can be simply packaged with the solution container. FIG. 15 shows an example of such an arrangement. As shown in the figure, a packaging system 420 includes a solution container 422 that can be provided in a package 424, such as a cardboard box. Also included in the package 424 is a package insert 426, such as a paper or cardboard insert, which includes multiple test areas 428 that contain a biosensor material. The solution stored in the container 422 can be tested as desired by squeezing a drop onto a given test area 428. As before, if the biosensor changes color, the solution is contaminated.

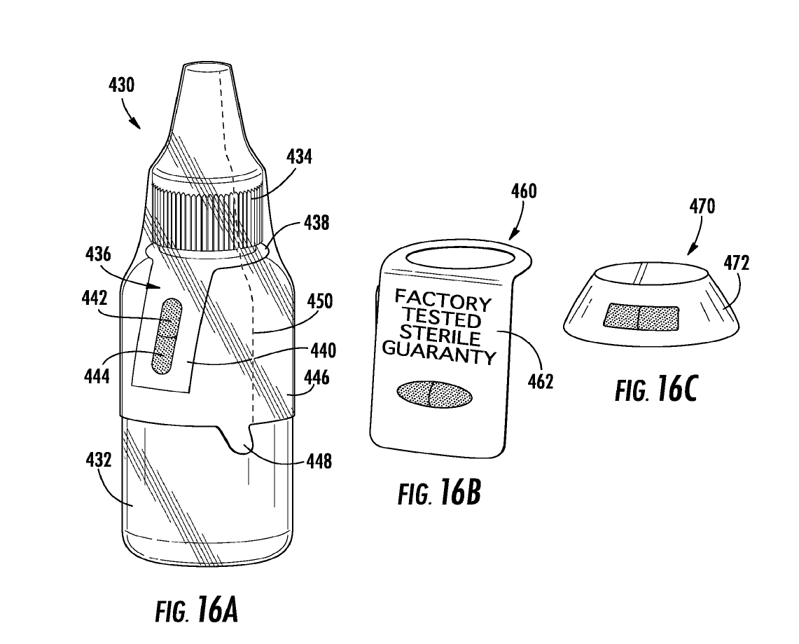

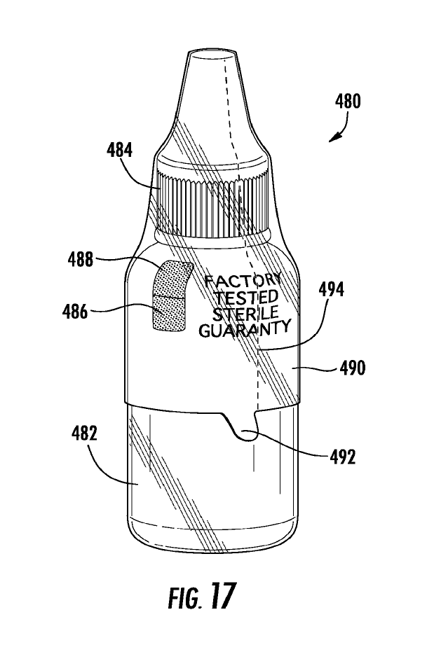

In further embodiments, the test area can be integrated with a safety collar that is provided on the solution container. FIGS. 16A-16C illustrate examples of such collars. Beginning with FIG. 16A, a solution container 430 comprises a body 432 and a cap 434. Provided around the neck of the body 432 below the cap 434 is a safety collar 436. In the embodiment of FIG. 16A, the collar 436 comprises a ring portion 438 that surrounds the neck and an elongated member or tongue 440 that extends downward from the ring portion. Biosensor material is provided on the tongue 440. In some embodiments, each test strip 408 comprises a control area 442 and a biosensor area 444 in similar manner to the like-named areas provided on the aforementioned test strips. As is further illustrated in FIG. 16A, at least the cap 434 and the safety collar 436 are wrapped in a clear tamper-resistant seal 446. In some embodiments, the seal 446 includes a pull tab 448 that can be used to remove the seal by tearing it along a perforation line 450.

During manufacturing, the manufacturer can apply a small sample of the solution used to fill the container 430 on the biosensor material provided on the tongue 440 of the safety collar 436. If the biosensor material does not change color at that time, the solution is safe to be shipped. In some instances, contaminants, such as bacteria, may slowly grow in the solution. In such a case, it is possible for the solution to pass testing at the factory but become contaminated to the point at which it should not be used at a later date. When the safety collar 436 is provided, the doctor or pharmacist who intends to provide the solution to a patient or the consumer who intends to buy the solution from a store can check the safety collar to confirm that no such contaminants have grown.

FIGS. 16B and 16C show alternative configurations for a safety collar that can be used with the solution container 430 shown in FIG. 16A. The safety collar 460 shown in FIG. 16B has a wider tongue 462 so as to provide more space for indicia to be provided on the collar. The safety collar 470 shown in FIG. 16C only comprises a ring portion 472, which can have a frustoconical shape.

FIG. 17 shows a solution container 480 that is a variation on the theme illustrated in FIGS. 16A-16C. In the embodiment of FIG. 17, the container 480 comprises a body 482 and a cap 484. Provided on an exterior surface of the body 482 near the cap 484 at the top portion of the body is a biosensor material. In the illustrated embodiment, the body 482 comprises a control area 486 and a biosensor area 488 that are similar to like-named areas described above. As is further illustrated in FIG. 17, at least the cap 484 and the areas 486, 488 are wrapped in a clear tamper-resistant seal 490, which includes a pull tab 492 that can be used to remove the seal by tearing it along a perforation line 494.

As expressed above, the embodiments disclosed herein are mere examples of the inventive subject matter. Accordingly, alternative embodiments are possible. Such alternative embodiments include embodiments that combine discrete features of the various embodiments explicitly described above.

1. A solution container comprising:
a body adapted to store a solution; and
a biosensor material integrated with the container, wherein the biosensor material has a first color when it has not been exposed to a contaminant but turns a second color when it comes into contact with a contaminant.

2. The container of claim 1, wherein the biosensor material comprises polydiacetylene (PDA).

3. The container of claim 1, wherein the biosensor material is integrated with the body of the container.

4. The container of claim 3, wherein the biosensor material is embedded within material used to form the body.

5.-8. (canceled)

9. The container of claim 3, wherein the biosensor material comprises a coating applied to an inner surface of the body.

10.-11. (canceled)

12. The container of claim 3, wherein the biosensor material comprises an element that is provided in the solution within the body.

13. The container of claim 3, wherein the body comprises an upper chamber adapted to store the solution and a lower chamber that contains the biosensor material, wherein the chambers are separated by a divider wall that includes a one-way valve that allows droplets of the solution to pass from the upper chamber to the lower chamber.

14. The container of claim 3, wherein the biosensor material is provided on a test strip that is associated with the body.

15.-17. (canceled)

18. The container of claim 1, further comprising a nozzle through which the solution can exit the body, wherein the biosensor material is integrated with the nozzle.

19.-22. (canceled)

23. The container of claim 18, wherein the container has an inverted configuration in which the nozzle faces downward.

24. The container of claim 1, further comprising a cap and wherein the body comprises a neck onto which the cap can be threaded, wherein the biosensor material is integrated with the cap.

25.-32. (canceled)

33. The container of claim 24, wherein the cap comprises an external well that contains the biosensor material.

34. The container of claim 24, wherein the cap is made of a clear material and comprises multiple internal cavities that contain the biosensor material.

35. (canceled)

36. The container of claim 1, further comprising a base member upon which the body can rest, the base member including at least one well that contains the biosensor material.

37. The container of claim 1, further comprising a safety collar attached to the body, the safety collar comprising the biosensor material.

38. (canceled)

39. A packaging system comprising:
a package;
a solution container adapted to fit within the package and store a solution; and
an insert adapted to fit within the package, the insert including multiple test areas that comprise a biosensor material that has a first color when it has not been exposed to a contaminant but turns a second color when it comes into contact with a contaminant.

40. A method for communicating the condition of a solution, the method comprising:
providing a container in which the solution is stored; and
providing a biosensor material integrated with the container, wherein the biosensor material has a first color when it has not been exposed to a contaminant but turns a second color when it comes into contact with a contaminant.

41. (canceled)
